(19) United States
(12) Patent Application Publication
(10) Pub. No.: US 2023/0043406 A1
(43) Pub. Date: Feb. 9, 2023

(54) NONINVASIVE METHOD AND SYSTEM FOR SLEEP APNEA DETECTION

(71) Applicant: SOUTH CHINA NORMAL UNIVERSITY, Guangzhou City (CN)

(72) Inventors: Han ZHANG, Guangzhou City, Guangdong Province (CN); Weiwei ZHU, Guangzhou City, Guangdong Province (CN); Songbin YE, Guangzhou City, Guangdong Province (CN); Baoxian YU, Guangzhou City, Guangdong Province (CN)

(21) Appl. No.: 17/790,445

(22) PCT Filed: Nov. 12, 2020
(86) PCT No.: PCT/CN2020/128426

§ 371 Date: Jun. 30, 2022

(30) Foreign Application Priority Data
Dec. 31, 2019 (CN) 201911413560.9

(51) Int. Cl. A61B 5/00

(52) U.S. Cl. CPC A61B 5/4818; A61B 5/7203; A61B 5/7267

(57) ABSTRACT

A noninvasive method and system for sleep apnea detection is disclosed. The method includes the following steps: acquiring vital sign signals of a sleeping user; performing structured processing on the vital sign signals of the user to remove invalid signals to obtain a set of valid vital sign signals; extracting multi-dimensional morphological features from a sleep respiratory signal and performing feature training on an initial model of a classifier by means of the multi-dimensional morphological features so as to obtain a sleep breathing detection model; and inputting the set of valid vital sign signals into the sleep breathing detection model and performing signal processing to obtain predicted probability of the user suffering from sleep apnea. As a result, data relating to the probability of a user suffering from sleep apnea can be more accurately obtained, thereby facilitating the determination of whether a sleep apnea event occurs during sleep.

### CROSS-REFERENCE TO RELATED APPLICATIONS

This application claims priority to Chinese Application No. CN 201911413560.9 having a filing date of Dec. 31, 2019, the entire contents of which are hereby incorporated by reference.

### FIELD OF TECHNOLOGY

The following relates to the technical field of sleep breathing signal, in particular to a noninvasive method and system for sleep apnea detection.

### BACKGROUND

Sleep is an important physiological activity that plays a key role in the physical and mental self-recovery of human beings. In recent years, with the acceleration of people's life rhythm and the increase of work pressure, people's awareness of their own health is increasing, and various portable medical testing equipment have been popularized in household applications. However, the general portable detection equipment processes the received signals of vital signs in a relatively simple manner, and its classification and detection methods are mostly limited to single-dimensional empirical statistics, not combined with multi-dimensional features to effectively train the signal detection model. Therefore, the classification accuracy may not be high, resulting in inaccurate detection of sleep apnea events.

### SUMMARY

To overcome the problems existing in related technologies, embodiments of the present disclosure provide a noninvasive method and system for sleep apnea detection.

According to a first aspect of an embodiment of the present disclosure, a noninvasive method for sleep apnea detection is provided, which includes the following steps:

acquiring vital sign signals of a sleeping user;

performing structured processing on the vital sign signals of the user to remove invalid signals to obtain a set of valid vital sign signals;

extracting multi-dimensional morphological features from a sleep respiratory signal and performing feature training on an initial model of classifier by means of the multi-dimensional morphological features so as to obtain a sleep breathing detection model; and

inputting the set of valid vital sign signals into the sleep breathing detection model and performing signal processing to obtain predicted probability of the user suffering from sleep apnea.

The noninvasive method for sleep apnea detection described in this embodiment employs multi-dimensional morphological features to perform feature training on an initial model of classifier to obtain a sleep breathing detection model, so that the performance of the sleep breathing detection model is strengthened and the predicted probability of a user suffering from sleep apnea can be more accurately obtained, thereby facilitating the accurate determination of a doctor or a user about whether a sleep apnea event occurs during sleep.

In an alternative embodiment, the step of performing structured processing on the vital sign signals of a user to remove invalid signals to obtain a set of valid vital sign signals further includes below steps:

removing out-of-bed signals by judgment method of out-of-bed;

removing body motion signals by judgment method of body motion;

removing invalid signal intervals by the signal validity determination; and

splicing the signals of the vital signs after removing the invalid signal intervals to obtain the set of valid vital sign signals without interference.

In an alternative embodiment, the step of extracting multi-dimensional morphological features from a sleep respiratory signal and performing feature training on an initial model of a classifier by means of the multi-dimensional morphological features so as to obtain a sleep breathing detection model further includes below steps:

performing structured processing on the sleep respiratory signal to remove invalid signal, and obtaining a set of valid sleep respiratory signal;

extracting a BCG sample signal from the valid sleep respiratory signal;

extracting a set of multi-dimensional morphological features of the BCG sample signal within a fixed time scale, the multi-dimensional morphological features include: low frequency feature, peak feature, area feature, power spectrum feature and nonlinear feature;

inputting an extracted set of multi-dimensional morphological features of the BCG sample signal into an ensemble learning model to optimize features to obtain a set of steady features; and

inputting the set of steady features into multiple initial models of classifier and performing feature classification training to obtain the sleep breathing detection model.

In an alternative embodiment, the step of extracting a BCG sample signal from the valid sleep respiratory signal further includes below steps:

identifying a J peak and a K valley of the BCG sample signal, and locating the J peak and the K valley in each BCG sample signal within a fixed time scale;

identifying the J peak to the left along a first time scale and the K valley to the right along a second time scale to locate a complete BCG sample signal and locate all BCG sample signals within the fixed time scale; and

selecting the BCG sample signal within the fixed time scale.

In an alternative embodiment, the step of selecting BCG sample signals within the fixed time scale further includes below steps:

calculating the mean value of all BCG sample signals in a fixed time scale to be used as a BCG sample signal model;

calculating the normalized Euclidean distance and normalized dynamic time warping distance between all BCG sample signals and the BCG sample signal model in fixed time scale; and

setting a default threshold of Euclidean distance and a default threshold of dynamic time warping, and discarding BCG signals whose normalized Euclidean distance is greater than the default threshold of Euclidean distance and whose normalized dynamic time warping distance is greater than the default threshold of dynamic time warping to obtain the BCG sample signal.

In an alternative embodiment, the step of inputting the extracted set of multi-dimensional morphological features of the BCG sample signal into an ensemble learning model to optimize features further includes below steps:

inputting a multi-dimensional morphological feature set into a tree model for sample feature training to obtain a first training loss;

performing random up-down permutation on specific columns in the multi-dimensional morphological feature set; after the up-down permutation of the specific columns, inputting the multi-dimensional morphological feature set into the tree model for sample feature training to obtain a second training loss;

calculating the difference between the values of the first training loss and the second training loss and the absolute value of the difference;

presetting an empirical threshold, deleting the corresponding morphological features whose absolute value of the difference between the first training loss and the second training loss is smaller than the preset empirical threshold, to obtain an optimized feature set; and

performing optimization training on the optimized feature set again to obtain a steady feature set.

In an alternative embodiment, the initial model of the classifier includes an LR classifier, a SVM classifier, a RF classifier and an AdaBoost classifier.

According to a second aspect of an embodiment of the present disclosure, a noninvasive system for sleep apnea detection is provided, which includes:

a vital sign signal acquisition device, for collecting the vital sign signals of a sleeping user;

a memory, for storing a program; and

a processor, for implementing the method described above by executing the program stored in the memory.

The noninvasive system for sleep apnea detection described in this embodiment of the present disclosure, collects the mysterious vital sign signals of a user during sleep through a portable, non-contact vital sign signal acquisition device, which gives the user a better test experience and will not affect the normal sleep of the user. In addition, the signal detection system can perform noise filtering on the vital sign signals of a user during sleep, and perform the signal processing and analysis more accurately, so that a user or a doctor can accurately determine whether a sleep apnea event occurs during sleep.

It should be understood that the above general description and the subsequent detailed description are only illustrative and explanatory but not limiting to the present disclosure.

For a better understanding and implementation, the disclosure will be described in detail below in combination with the accompanying drawings.

### BRIEF DESCRIPTION OF THE DRAWINGS

FIG. 1 is a flowchart illustrating of a noninvasive method for sleep apnea detection according to an embodiment of the present disclosure;

FIG. 2 is a flowchart illustrating of the step of S2 of a noninvasive method for sleep apnea detection according to an embodiment of the present disclosure;

FIG. 3 is a flowchart illustrating of the step of S3 of a noninvasive method for sleep apnea detection according to an embodiment of the present disclosure.

### DETAILED DESCRIPTION

Exemplary embodiments will be described in detail herein, examples of which are illustrated in the accompanying figures. Where the following description refers to the drawings, the same numerals in different figures refer to the same or similar elements unless otherwise indicated. The implementations described in the exemplary embodiments below are not intended to represent all implementations consistent with the disclosure. Rather, they are merely examples of apparatus and methods consistent with some aspects of the application as recited in the appended claims.

The terminology used herein is for the purpose of describing particular embodiments only and is not intended to be limiting of the present disclosure. As used herein, the singular forms “a,” “an,” and “the” are intended to include the plural forms as well as the singular forms, unless the context clearly indicates otherwise. It will be further understood, the term “and/or” includes any and all combinations of one or more of the associated listed items.

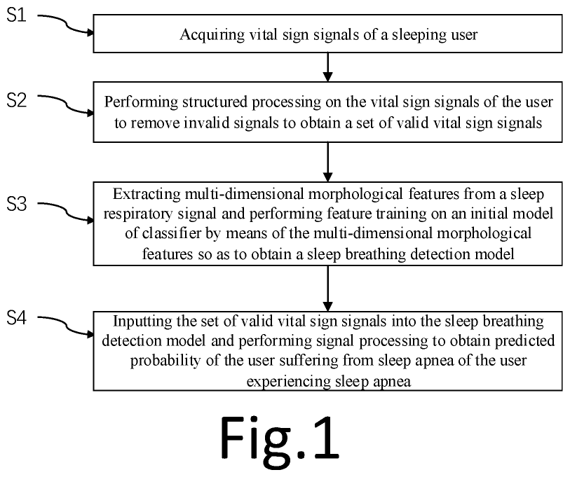

Please refer to FIG. 1, which is a flowchart illustrating of a noninvasive method for sleep apnea detection according to some embodiment of the present disclosure.

The detection method for sleep apnea assessment in this embodiment includes the following steps:

S1: Acquiring vital sign signals of a sleeping user;

S2: Performing structured processing on the vital sign signals of the user to remove invalid signals to obtain a set of valid vital sign signals;

S3: extracting multi-dimensional morphological features from a sleep respiratory signal and performing feature training on an initial model of classifier by means of the multi-dimensional morphological features so as to obtain a sleep breathing detection model; and

S4: Inputting the set of valid vital sign signals into the sleep breathing detection model and performing signal processing to obtain predicted probability of the user suffering from sleep apnea.

The noninvasive method for sleep apnea detection described in this embodiment of the present disclosure, employs multi-dimensional morphological features extracted from a sleep respiratory signal to perform feature training on an initial model of classifier to obtain a sleep breathing detection model, and inputs a user's vital sign signals after structured processing into the sleep breathing detection model to obtain more accurate predicted probability of a user suffering from sleep apnea, so that a user or doctor can precisely determine a sleep apnea event occurs during sleep.

Since there are signal noises such as out-of-bed signals, body motion signals, and other invalid signals in the collected signals in a continuous time period, it is necessary to remove the above invalid signals to make the signals collected useful.

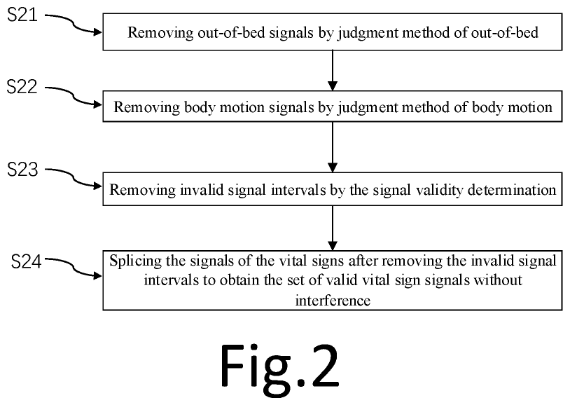

Accordingly please refer to FIG. 2, which is a flowchart illustrating of the step of S2 of the noninvasive method for sleep apnea detection according to some embodiment of the present disclosure.

In step S2, wherein the step of performing structured processing on the vital sign signals of a user to remove invalid signals to obtain a set of valid vital sign signals, further comprises:

S21: Removing out-of-bed signals by judgment method of out-of-bed.

Specifically, when the user is out of bed, the collected signals only include thermal noise, which are invalid and need to be removed. Therefore, the judgment method of out-of-bed is as follows: according to the statistical characteristics of Gaussian white noise, a time scale T can be defined to analyze the sleep breathing signal within the time scale T, and the mathematical expectation, power spectral density, autocorrelation and signal amplitude of the sleep breathing signal within the time scale can be obtained; When the mathematical expectation is approximate to zero, the power spectral density is approximate to constant, the autocorrelation of the time domain signal is approximate to impact, and the signal amplitude is less than the preset fixed threshold, the sleep breathing signal of the user at this time is determined to be out-of-bed, which is to be removed.

S22: Removing body motion signals by judgment method of body motion.

Specifically, it includes the big body motion signals removal and the small body motion signals removal.

The removal of the big body motion signals is as follows: within a fixed time scale T, the start and end times of big body motion are determined according to a preset limit threshold value of the signal, and the amplitude of the limit threshold is adjusted to determine the big body motion signal, which is to be removed.

The removal of the small body motion signals is as follows: the envelope function mu(t) in the sleep breathing signal is calculated with Hilbert transform, the ratio of the maximum value max { } to the minimum value min{ } in the envelope function, and the ratio of the maximum value max{ } to the mean value mean{ } are calculated; when max{mu(t)}/min{mu(t)}>p1 and max{mu(t)}/mean{mu(t)}>p2, where p1 and p2 are the empirical thresholds respectively, it is determined that there is a small body motion signal in this time scale; the amplitude of the limit threshold is adjusted to determine the small body motion signal, which is to be removed.

S23: Removing invalid signal intervals by the signal validity determination.

When the distance between the user's body and the sensor is too far, the SNR of the output signal is too low, which is not useful for analysis. In this case, the approximate periodicity of cardiac impact diagram and respiratory signal is drowned by noise, so the invalid signal interval needs to be removed.

Specifically, the determination of the validity of the signal is as follows: through empirical mode decomposition and wavelet transform, smooth circulation characteristics of the signal of the vital signs within the fixed time scale Tin different frequency interval is analyzed with autocorrelation, then the sleep breathing signal within the time scale is determined valid or not, and the invalid signal corresponding to the interval is removed.

S24: Splicing the signals of the vital signs after removing the invalid signal intervals to obtain a set of valid vital sign signals without interference.

In some embodiments, the following steps are further included before splicing the sleep respiratory signals reasonably: a first-order statistics and a second-order statistics of the signals in the adjacent interval are counted respectively; when the first-order statistics and the second-order statistics of the signals in the adjacent interval are both lower than the preset fixed threshold, the data are directly combined and spliced; when the first-order statistics and the second-order statistics of the signals in the adjacent interval are not less than the preset fixed threshold, the two signals are classified and determined respectively.

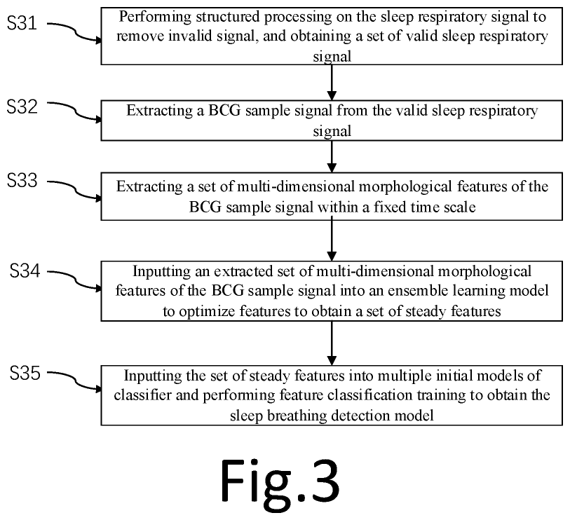

Please refer to FIG. 3, which is a flowchart illustrating of the step of S3 of the noninvasive method for sleep apnea detection according to some embodiment of the present disclosure.

In S3 that multi-dimensional morphological features are extracted from the obtained sleep respiratory signal to train an initial model of a classifier and a sleep breathing detection model is obtained, there are following steps further included:

S31: Performing structured processing on the sleep respiratory signal to remove invalid signal and obtaining a set of valid sleep respiratory signal;

S32: Extracting a BCG sample signal from the valid sleep respiratory signal;

S33: Extracting a set of multi-dimensional morphological features of the BCG sample signal within a fixed time scale, the multi-dimensional morphological features include: low frequency feature, peak feature, area feature, power spectrum feature and nonlinear feature;

S34: Inputting an extracted set of multi-dimensional morphological features of the BCG sample signal into an ensemble learning model to optimize features to obtain a set of steady features; and

S35: Inputting the set of steady features into multiple initial models of classifier and performing feature classification training to obtain the sleep breathing detection model.

Wherein, S32 further includes below steps:

identifying a J peak and a K valley of the BCG sample signal, and locating the J peak and the K valley in each BCG sample signal within a fixed time scale;

identifying the J peak to the left along a first time scale and the K valley to the right along a second time scale to locate a complete BCG sample signal and locate all BCG sample signals within the fixed time scale; and

selecting the BCG sample signal within the fixed time scale.

In an embodiment, BCG sample signals are selected by following steps:

calculating the mean value of all BCG sample signals in a fixed time scale to be used as a BCG sample signal model;

calculating the normalized Euclidean distance and normalized dynamic time warping distance between all BCG sample signals and the BCG sample signal model in fixed time scale;

and

setting a default threshold of Euclidean distance and a default threshold of dynamic time warping, and discarding BCG signals whose normalized Euclidean distance is greater than the default threshold of Euclidean distance and whose normalized dynamic time warping distance is greater than the default threshold of dynamic time warping to obtain the BCG sample signal.

In some embodiment, in the step of S33, multi-dimensional morphological features are extracted by following methods:

For Low Frequency Feature:

Within the fixed time scale, according to the identification of the J peak and K valley of the BCG sample signal, the information between the J peak and the K valley is extracted, and the upper and lower envelope functions in the time window are obtained through multiple spline interpolation, and the upper and lower envelope functions are subjected to empirical mode decomposition to extract the low-frequency components in the upper and lower envelope functions, which are defined as Eu(t) and Ed(t). Specifically, the upper and lower envelopes cover the J peak and K valley of the BCG signal, the upper envelope is defined as the function mu(t), and the lower envelope is defined as the function md(t). Empirical mode decomposition is performed on mu(t) and md(t) respectively to extract low frequency parts of the upper and lower envelope function which are defined as Eu(t) and Ed(t) respectively. Since low frequency parts can truly reflect original volatility of signals, the volatility and complexity of Eu(t) and Ed(t) can be used as one of the features to determine apnea.

For Peak Feature:

The J peak and K valley of each BCG sample signal in a fixed time scale are identified to form sets of {CJ(i)} and {CK(i)}, where i is the number of BCG signals in the current time scale.

Because the morphological characteristics from J peak to K valley in BCG sample signal are the most robust and subject to the fluctuation and oscillation effect of respiratory wave, the variance, standard deviation, kurtosis and slopes of {CJ(i)} and {CK(i)} sets are also calculated, which are used as features to identify apnea.

In addition, the first-order difference of the adjacent data in the sets of {CJ(i)} and {CK(i)} is calculated to reconstruct the new sets of {ΔCJ(i)} and {ΔCK(i)}, and the second-order difference is further solved to construct the new sets of {Δ2CJ(i)} and {Δ2CK(i)}. One-dimensional and two-dimensional data statistics are performed on the first-order difference and second-order difference sets of the data sets respectively, and numerical variance and standard deviation are calculated, which are used as features to identify apnea.

For Area Feature:

The integral of coverage area of H peak, I valley, I peak, J peak, K valley and L peak of each BCG sample signal in the fixed time scale are calculated, i.e., the area of each BCG signal from the H peak to the L peak, and the variance and standard deviation of the envelope of coverage area of the BCG sample signal are calculated, which are used as features to identify apnea.

For Power Spectrum Feature:

A fixed-length Fourier transform for each complete BCG signal is solved. The ratio of high and low frequency of power spectral density of adjacent BCG signals are quantified. All these results and the low frequency fluctuation of all BCG signal power spectra in a fixed time scale are regarded as features to identify apnea.

For Nonlinear Feature:

The original signal at a fixed time scale is processed by frequency reduction, and then the sample entropy is calculated and obtained. Entropy is a measure of the uncertainty of a random variable, and the greater the uncertainty, the greater the entropy. In this embodiment, the sample entropy is used, and the calculating of the sample entropy with other entropy values, such as approximate entropy, has two advantages: firstly, the sample entropy processing operation does not need to consider the length of data; secondly, the sample entropy has a good consistency. The smaller the value of sample entropy operation is, the higher the similarity of the sequence itself. On the contrary, the larger the value of sample entropy operation, the more chaotic and complex the sample sequence itself is. Therefore, sample entropy has been well used in assessing the degree of disorder with time-series physiological signals and determining pathology to distinguish numerical difference between paused and normal breathing segments.

In some embodiment, in the step of S34 of inputting the extracted set of multi-dimensional morphological features of the BCG sample signal into an ensemble learning model for features optimization, following steps are further included:

inputting a multi-dimensional morphological feature set into the tree model for sample feature training to obtain a first training loss e1, i.e., the first training loss e1 is obtained by training based on the feature samples extracted for the first time for any tree model Qi, i=1, 2, . . . , N, where N is the number of trees in the model;

performing random up-down permutation on specific columns in the multi-dimensional morphological feature set; after the up-down permutation of the specific columns, inputting the multi-dimensional morphological feature set into the tree model for sample feature training to obtain a second training loss; specifically, other columns unchanged, performing random up-down permutations on the jth column, i.e., feature j, to obtain the second training loss e2;

calculating the difference between the values of the first training loss and the second training loss and the absolute value of the difference |e1−e2|;

presetting an empirical threshold, deleting the corresponding morphological features whose absolute value of the difference between the first training loss and the second training loss is smaller than the preset empirical threshold, to obtain an optimized feature set, i.e., an empirical threshold 13 is set, when feature importance |e1−e2|<β, it is defined that the feature has a limited contribution to the overall decision of the model or has a negative contribution, so the feature is deleted; and

performing optimization training on the optimized feature set again to obtain a steady feature set.

Specifically, in the step of S4 to obtain probability of the user suffering from sleep apnea, the related classification decision method is as follows: with the help of the optimized feature set, N (N>1) classifiers including LR, SVM, RF, AdaBoost, etc., are used to classify apnea; combined with the statistical independence of the training loss of the output results of the different classifiers to a certain extent, the output results of all the classifiers are weighted to vote, i.e., the ratio is combined, and the binary decision of the apnea events is finally output, so as to facilitate the detection of infrequent short-term abnormal segments in long-term sign signals. The weighted coefficient is inversely proportional to the training loss coefficient of the training set.

In an optional embodiment, in order to verify the accuracy of the classification results of the classifiers and reduce misjudgment and missed detection in the preliminary classification, quantitative analysis based on different time scales is also carried out on the predicted probability to determine whether there is a fluctuation drop in the fragment signal within the corresponding time scale of the predicted probability. If there is a decrease in the fluctuation of the segment signal in the corresponding time scale, it indicates that the segment signal is a sleep apnea signal, which can verify that the predicted probability obtained by the sleep breathing detection model is relatively precise.

The quantitative analysis based on different time scales is: S={S1, S2, . . . , SN} as the apnea signal segment based on the preliminary detection of the classifier is defined, wherein Sn=[sn(t), sn(t+1), sn(t+L)], L is the pause time length, N is the number of paused segments detected by the classifier, and sn(t+1) is the signal corresponding to the paused segment at time t+1. {T} is defined as a variable time scale, for example: T=30 s, 60 s, 120 s, and whether the apnea signal segment actually has a trend of “declining volatility” at different time scales {T} is calculated, while the specific detection method is based on multi-scale normalized mean absolute deviation analysis or multi-scale normalized variance analysis.

Wherein, the multi-scale normalized mean absolute deviation analysis is as follows: the MAD (mean absolute error) value of the signal under a certain time scale is calculated and the result is defined as MT; time window is slid in 10 s in the time scale, each MAD value corresponding to every 10 s sliding in the time window is calculated, the set of MAD under the corresponding time scale T is defined as M=[M1, M2, . . . , M(T−10)], and c_{T}=min{M}/M_{T} is calculated. The volatility obtained by the analysis of the smaller time scale is the smaller volatility, and the volatility obtained by the analysis of the larger time scale is the larger volatility, and the MAD value can well describe the fluctuation degree of the signal. After the predicted probability obtained from the breathing detection model, the fluctuation of the sleep apnea signal segment at different time scales is analyzed, wherein the result can reflect whether the apnea signal segment has a significant decrease in volatility compared with the normal breathing segment. When the ratio of the minimum volatility to the average volatility is less than 0.2, i.e., when cT<0.2, it means that the apnea signal segment has a significant decrease in volatility relative to the normal breathing segment, which can verify the accuracy of the classification.

In addition, the multi-scale normalized variance analysis can also better describe the degree of signal stability and alternation, which calculate the ratio of normalized variance between apnea signal fragments and normal respiratory signal fragments under multi-scale. That can also verify the accuracy of the classification.

The embodiment of the present disclosure discloses a noninvasive method for sleep apnea detection. Vital sign signals are collected by a piezoelectric sensor, and the collected vital sign signals are subjected to structured processing to remove motion noise and other noises, and then the set of valid sign signals is input into the sleep breathing detection model trained based on multi-dimensional morphological features for signal processing, and the predicted probability of apnea occurring during sleep is obtained, so that users or doctors can more accurately determine whether apnea occurred during sleep and the time period during which the apnea event occurred. This method can be practically referenced in pre-examination of sleep apnea at home outside the hospital in the future.

The embodiment of the present disclosure also discloses a noninvasive system for sleep apnea detection, which includes: a vital sign signal acquisition device, for collecting the vital sign signals of a sleeping user; a memory, for storing a program; and a processor, for implementing the method described above by executing the program stored in the memory.

The vital sign signal acquisition device is a piezoelectric sensor module. During signal acquisition, the user just needs to place the piezoelectric sensor module under the user's head to observe the user's sleep and breathing for a long time, while the user can sleep normally without any interference.

In an embodiment, the noninvasive system for sleep apnea detection further includes an A/D conversion module, a buffer module and a filter module, which are connected sequentially to the signal output end of the vital sign signal acquisition device; the A/D conversion module converts vital sign signals from analog signals to digital signals; the input of the buffer module is the vital sign digital signals after A/D conversion, and the output of the buffer module is the stack signals to be processed; the filter module includes three filtering combinations of low-pass filtering, band-pass filtering, and morphological filtering, wherein low-pass filtering removes high-frequency noise, band-pass filtering separates vital signs signals of each frequency band, and morphological filtering identifies the signal baseline value and low-frequency fluctuation characteristics and removes signal baseline interference.

In the noninvasive system for sleep apnea detection according to the embodiment of the present disclosure, the vital sign signals of a user during sleep are obtained through a portable vital sign signal acquisition device, while the user does not need to wear electrodes and feels more comfortable during the test; meanwhile the vital sign signals are subjected to structured processing to remove signal noise, before they are input into the signal detection model for signal processing to obtain the predicted probability of the user suffering from sleep apnea, so as to improve the accuracy of the determination of whether a sleep apnea event occurs during sleep. In the signal processing, the system performs feature training on an initial model of a classifier through the set of multi-dimensional morphological features of sleep respiratory signals, so as to strengthen the performance of a resulting trained classifier model. This system can be practically referenced in pre-examination of sleep apnea at home outside the hospital in the future, which is very convenient for a user to obtain long-term measurements outside the hospital.

The above embodiments are only used to illustrate the implementation of the present disclosure, but not to limit it. While the present application has been described with reference to above embodiments in detail, it will be understood by those skilled in the art that various changes may be made and equivalents may be substituted for elements thereof without departing from the scope of the present disclosure.

1. A noninvasive method for sleep apnea detection, comprising steps of:
acquiring vital sign signals of a sleeping user;
performing structured processing on the vital sign signals of the user to remove invalid signals to obtain a set of valid vital sign signals;
extracting multi-dimensional morphological features from a sleep respiratory signal and performing feature training on an initial model of classifier by means of the multi-dimensional morphological features so as to obtain a sleep breathing detection model; and
inputting the set of valid vital sign signals into the sleep breathing detection model and performing signal processing to obtain predicted probability of the user suffering from sleep apnea;
wherein the step of extracting multi-dimensional morphological features from a sleep respiratory signal and performing feature training on an initial model of a classifier by means of the multi-dimensional morphological features so as to obtain a sleep breathing detection model comprises:
performing structured processing on the sleep respiratory signal to remove invalid signal, and obtaining a set of valid sleep respiratory signal;
extracting a BCG sample signal from the valid sleep respiratory signal;
extracting a set of multi-dimensional morphological features of the BCG sample signal within a fixed time scale, the multi-dimensional morphological features include: low frequency feature, peak feature, area feature, power spectrum feature and nonlinear feature;
inputting an extracted set of multi-dimensional morphological features of the BCG sample signal into an ensemble learning model to optimize features to obtain a set of steady features; and
inputting the set of steady features into multiple initial models of classifier and performing feature classification training to obtain the sleep breathing detection model;
wherein the step of extracting a BCG sample signal from the valid sleep respiratory signal comprises:
identifying a J peak and a K valley of the BCG sample signal, and locating the J peak and the K valley in each BCG sample signal within a fixed time scale;
identifying the J peak to the left along a first time scale and the K valley to the right along a second time scale to locate a complete BCG sample signal and locate all BCG sample signals within the fixed time scale; and
selecting the BCG sample signal within the fixed time scale.

2. The noninvasive method for sleep apnea detection of claim 1, wherein the step of performing structured processing on the vital sign signals of a user to remove invalid signals to obtain a set of valid vital sign signals comprises:
removing out-of-bed signals by judgment method of out-of-bed;
removing body motion signals by judgment method of body motion;
removing invalid signal intervals by the signal validity determination; and
splicing the signals of the vital signs after removing the invalid signal intervals to obtain the set of valid vital sign signals without interference.

3. (canceled)

4. (canceled)

5. The noninvasive method for sleep apnea detection of claim 1, wherein the step of selecting the BCG sample signal within the fixed time scale comprises:
calculating the mean value of all BCG sample signals in a fixed time scale to be used as a BCG sample signal model;
calculating the normalized Euclidean distance and normalized dynamic time warping distance between all BCG sample signals and the BCG sample signal model in fixed time scale; and
setting a default threshold of Euclidean distance and a default threshold of dynamic time warping, and discarding BCG signals whose normalized Euclidean distance is greater than the default threshold of Euclidean distance and whose normalized dynamic time warping distance is greater than the default threshold of dynamic time warping to obtain the BCG sample signal.

6. The noninvasive method for sleep apnea detection of claim 1, wherein the step of inputting the extracted set of multi-dimensional morphological features of the BCG sample signal into an ensemble learning model to optimize features comprises:
inputting a multi-dimensional morphological feature set into a tree model for sample feature training to obtain a first training loss;
performing random up-down permutation on specific columns in the multi-dimensional morphological feature set; after the up-down permutation of the specific columns, inputting the multi-dimensional morphological feature set into the tree model for sample feature training to obtain a second training loss;
calculating the difference between the values of the first training loss and the second training loss and the absolute value of the difference;
presetting an empirical threshold, deleting the corresponding morphological features whose absolute value of the difference between the first training loss and the second training loss is smaller than the preset empirical threshold, to obtain an optimized feature set; and
performing optimization training on the optimized feature set again to obtain a steady feature set.

7. The noninvasive method for sleep apnea detection of claim 1, wherein the initial model of the classifier comprises:
an LR classifier;
a SVM classifier;
a RF classifier; and
an AdaBoost classifier.

8. (canceled)
